SEQUENCE LISTING (3 sequences)

SEQ ID NO: 1 - DNA, 170 bp, Artificial Sequence
  Synthetic
atttgaagcgtcttaacgctcccaaagcatggatgttggacaagctgggcggcgtgtacgcgccgagaccctcgactggtcctcacaaacttcgcgagtgcttgccgctggtgatttttctgcgcaaccggctaaagtacgcgctcaccggtaatgaggtgctcaagatc

SEQ ID NO: 2 - DNA, 200 bp, Artificial Sequence
  Synthetic
gagcgtgccggccgtgtggagggcaggaacggctccatcacccagatccccattcttactatgcccaacgacgacatcacccatcctattcctgacttgacgggttatattactgagggacagatctacgtagaccgtcagctccacaacagacagatctacccgccagtgaacgtgctgccctctctgtcacgtttgat

SEQ ID NO: 3 - DNA, 200 bp, Artificial Sequence
  Synthetic
cctgaaatatttactgaaatctttggctgatgaaggaaatgtggaaaaaattcaacaattgggcaaatacattaccgatgcacagaaacggaatgtgacatacgacgataagttaacaaaggctatattcgcgagaggcgctgggtcacaacatgttgacaatttactacttacaataaaatcgaccaaatcagaagagg

(19) United States
(12) Patent Application Publication
(10) Pub. No.: US 2021/0321616 A1
(43) Pub. Date: Oct. 21, 2021

(54) PROCESS OF SELECTION OF A NUCLEOTIDE SEQUENCE FOR USE AS A PHYTOSANITARY PRODUCT, PLASMID OR COSMID, HOST CELL, PROCESS FOR THE PRODUCTION OF AN RNA MOLECULE, RNA MOLECULE, USE OF AN RNA MOLECULE, STABILIZED COMPOUND, PHYTOSANITARY PRODUCT AND METHOD TO ELIMINATE OR REDUCE THE INFESTATION OF AN INSECT, DISEASE OR WEED IN AGRICULTURE

(71) Applicant: Evolutta Agro Biotecnologia Ltda, Maringa PR (BR)

(72) Inventors: Sérgio Ricardo Nozawa, Ribeirão Preto (BR); Fabiano dos Santos Ferreira, Ribeirão Preto (BR)

(21) Appl. No.: 16/852,059

(22) Filed: Apr. 17, 2020

(51) Int. Cl. A01N 57/16; G16B 40/00; G16B 30/00; C12N 15/63

(52) U.S. Cl. CPC A01N 57/16; C12N 15/63; G16B 30/00; G16B 40/00

(57) ABSTRACT

This invention is related to a method of selection of a nucleotide sequence useful as a phytosanitary product. It also refers to a modified plasmid or cosmid comprising this nucleotide sequence, a host cell for its expression, the dsRNA molecule useful as phytosanitary product and compounds and/or phytosanitary products comprising it, to be used to eliminate or reduce the infestation of an insect, disease or weed in cultivated crops.

### SEQUENCE LISTING

The instant application contains a Sequence Listing which has been filed electronically in ASCII format and is hereby incorporated by reference in its entirety. Said ASCII copy, created on Apr. 15, 2020, is named 314441 US_SL.txt and is 2 KB in size.

### Technical Field of Invention

This invention is related to a method of selection of a nucleotide sequence useful as a phytosanitary product. It also refers to a modified plasmid or cosmid comprising this nucleotide sequence, a host cell for its expression, the dsRNA molecule useful as phytosanitary product and compounds and/or phytosanitary products comprising it, to be used to eliminate or reduce the infestation of an insect, disease or weed in cultivated crops.

### DESCRIPTION OF THE STATE OF THE ART

From planting to harvesting, cultivated crops are infested by a series of pests and diseases that attack the roots, stems, leaves and fruits. Agricultural crops are also infested by weeds, which compete for water, nutrients and light, and are hosts of insect pests and plant diseases.

An example of insect pest is the fall armyworm (Spodoptera frugiperda), considered the most harmful to corn crop, which attacks the plants both in the vegetative and reproductive stages, in high incidence and in high severity, being difficult to manage and causing high yield losses. It is a voracious pest of a polyphagous habit, having been reported almost 100 host species, from 27 families, with preference for grasses such as corn, millet, sorghum, rice and sugarcane (Goergen et al., 2016). In addition, the fall armyworm causes damage in other species of economic importance, such as soybeans, cotton, alfalfa, peanuts, sweet potatoes, eggplant, brassicas, onion, clove, chrysanthemum, cucurbitaceous, beans, geraniums, horticulture, pastures, pepper, tobacco, tomato, among others (EPPO, 2018).

The control of fall armyworm in corn has been done through the combination of transgenic corn (which expresses insecticide proteins) and the application of chemical insecticides, at a cost of billions of dollars per year, which should be accounted together with the loss of revenue resulting from the yield decrease caused by this insect.

This insect, however, has developed resistance to insecticides and transgenic plants available on the market, requiring a high number of additional applications of insecticides on crops, resulting in increased production costs, loss of yield and adverse effects on human health and the environment.

The Brazilian branch of the Insect Resistance Action Committee (IRAC-BR, 2013) reported the high risk of resistance of the fall armyworm (Spodoptera frugiperda) to insecticides and Bt corn due to biological factors such as its polyphagous habit, high reproductive potential, high dispersal capacity and due to climatic and environmental factors, such as temperature and humidity conditions favorable to the development of this species throughout the year, in various regions of Brazil.

Insect resistance monitoring projects conducted by RAC (2019) since the 2014/2015 season, in all farming regions in Brazil, with the most important chemical insecticides of different modes of action, showed increased survival of caterpillars treated with lambda-cyhalothrin, chlorpyrifos and thiodicarb. The active ingredients novaluron, chlorfluazuron, spinosad, indoxacarb, clorantraniliprole and flubendiamide, still result in the survival of caterpillars at levels below those considered critical for insect resistance. Factors such as the intensive use of chemical insecticides for pest control, including fall armyworm, the low adoption of Insect Resistance Management (IRM) practices and the increase in the number of sprays during the crop cycle, have made intense selective pressure to resistant biotypes, which will lead, in the near future, to great difficulties in chemical pest control in crops.

In transgenic corn (Bt), due to the extensive area of cultivation, the low adoption of the planting of refuge areas and the similarity of the modes of action, transgenic proteins have been rapidly losing their effectiveness. Studies of fall armyworm populations conducted in Brazil, sampled in several corn producing regions, detected a decrease in the susceptibility of this species to Cry1F protein expressed by genetically modified Bt corn plants (Farias et al., 2014). In turn, Burtet et al. (2017) reported that Bt corn hybrids (YieldGard VT Pro, YieldGard VT Pro 3, PowerCore, Agrisure Viptera and Agrisure Viptera3), in late plantings in southern Brazil, were not effective enough to control the infestation of fall armyworm and required the spraying of insecticides. Omoto et al. (2016) identified populations of fall armyworm with reduced susceptibility to cry1Ab transgenic protein expressed in Bt corn hybrids.

As with Spodoptera frugiperda, the vast majority of the pest species that infest crops are developing resistance to insecticides and transgenic plants, so that in the near future, can make food production economically inviable in the main producing regions of the world with a high impact on food security, if new compounds and new technologies are not developed and made available for the control of agricultural pests.

Thus, it is urgent to develop new pest control methods that have action on specific metabolic functions related to the development and/or reproduction of pests, but without presenting the undesired effects of traditional agrochemicals, such as effect on non-target organisms, high toxicity, runoff effects, water contamination, exposure of applicators, among others.

In this context, RNA interference (RNAi) has been deeply studied as an effective and environmentally safe alternative for the control of insects, diseases and/or weeds in crops, through double-stranded RNA products (dsRNA).

RNAi is a natural mechanism in cells of superior organisms (eukaryotes) with important functions such as the control of gene expression and the defense against viral infections. For the activation of a gene, cells transcribe a certain DNA sequence into a messenger RNA (mRNA) that is transported from the nucleus to the cytoplasm, where it is translated into a protein. Similarly, cells need to control the intensity of the expression of their genes, reducing or silencing their expression through the RNAi mechanism, depending on the body's need for a certain protein.

The RNAi mechanism can be divided into three basic steps: initially the presence of a double-stranded RNA molecule (dsRNA) activates the cutting mechanism of this RNA in small double-stranded RNAs (siRNA) by the action of an enzyme called Dicer (ribonuclease III). Then, one of the siRNA strands is removed and the remaining strand is incorporated into a protein complex called RNA Induced Silencing Complex (RISC). Finally, the RISC complex identifies and degrades messenger RNA (mRNA) that have the same sequence as siRNA, preventing protein synthesis.

This process ensures high specificity of the RNAi mechanism, so that any mRNA of a cell can be intentionally degraded by RNAi. In addition, once it is activated in one cell, the RNAi signal is transmitted to the other cells, exponentially increasing its effect.

The high specificity of the dsRNA mechanism makes this technology safe for the environment and human health, with no effect on non-target organisms, since gene sequences of both the pest and non-target species have variations that allow scientists to engineer specific dsRNA with action only on the targets. In addition, the technology allows a great predictability of its action, since it is possible to compare the sequence of the engineered dsRNA with the hundreds of thousands of DNA sequences present in global databases, and it can be verified, beforehand, which species can be affected by dsRNA and change its sequence until only the desired targets are affected.

However, despite being considered highly effective and safe, dsRNA-based products are not available to farmers due to technical barriers related to the nature of the RNA molecule, which makes it highly unstable and rapidly degraded by environmental factors such as light, temperature and pH, making it impossible to develop and commercialize a product based on this active ingredient to be sprayed on crops under field conditions.

In addition, the high cost of the synthesis of dsRNA molecules, usually made through virus culture, which can reach thousands of dollars per few grams, makes large-scale manufacturing processes economically inviable to produce affordable products for farming use.

### Objectives of the Invention

The first objective of this invention is to provide a process for the selection of a nucleotide sequence useful for use as a phytosanitary product for the control of insects, diseases and weeds, which is effective and safe.

The second objective is the description of a plasmid or cosmid, which comprises the selected sequence to be expressed, being able to optimize which targets to be inactivated.

In this same reasoning, therefore, the third objective of this invention is the description of a host cell that comprises the modified plasmid or cosmid, which is capable of efficiently expressing the selected nucleotide sequence.

The fourth objective of the this invention is to describe a process for the production of an RNA, particularly an interference RNA (RNAi), especially a double-stranded RNA (dsRNA), which is efficient and low cost, in order to economically enable the use of RNA as a phytosanitary product for application in crops.

Thus, fifth and sixth objectives present here, are the RNA molecule obtained by the method proposed here and its use as a phytosanitary product, respectively.

The seventh and eighth objectives of the invention, respectively, are the description of a stabilized compound that comprises the produced RNA molecule, and a phytosanitary product that comprises the RNA molecule or the stabilized compound.

Finally, the ninth objective the description is a method to eliminate or reduce the infestation of an insect, disease or weed in cultivated crops.

### BRIEF DESCRIPTION OF THE INVENTION

The first objective is achieved through a process of selecting a nucleotide sequence for use as a phytosanitary product comprising the following steps:
a. analysis of the transcriptome of the target pest;
b. selection of the sequence of a low-expression gene, which is more sensitive to silencing, but which has participation in essential metabolic pathways, physiological and/or reproductive processes;
c. determination of homology and similarity of the selected sequence (in step b) with non-target organisms; and
d. selection of the target nucleotide sequence by means of a Decision Tree algorithm to identify the sequence of high metabolic performance and low similarity or homology with non-target organisms.

The second objective consists of a plasmid or cosmid comprising at least one copy of the nucleotide sequence selected by the process as defined above.

The third objective is achieved by a host cell comprising at least one of the plasmids or cosmid, and that its cellular machinery has tools that allow its expression.

The fourth objective is achieved by a process to produce an RNA comprising the following stages:
a. selection of a nucleotide sequence;
b. preparation of a plasmid or cosmid;
c. transfection of a host cell with this plasmid;
d. multiplication of host cells in a bioreactor, and
e. expression of the selected nucleotide sequence.

Therefore, the fifth objective comprises an RNA molecule obtained by the process described above.

The sixth objective is achieved using the RNA molecule as a phytosanitary product to eliminate or reduce the infestation of an insect, disease or weed in crops.

The stabilized compound of the seventh objective is achieved through a compound comprising the RNA molecule in combination with one or more stabilizing agents.

On the other hand, the phytosanitary product of the eighth objective comprises the RNA molecule or a stabilized compound.

Finally, the ninth objective is achieved by a method to eliminate or reduce the infestation of an insect, disease or weed in the crop, which includes spraying this phytosanitary product over the infested area or the leaf surface.

### DESCRIPTION OF THE FIGURES

FIG. 1—Decision Tree model for the selection of dsRNA genes based on their level of expression, which will be synthesized, cloned in cosmid and inserted into a genetically modified E. coli for the large-scale production of stabilized dsRNA for use as a phytosanitary product.

FIG. 2—Test results with 25 buffers to measure buffering efficiency, molecule activity and resulting pH.

FIG. 3—Cosmid EVO-206910. In Polylinker: Ori (Origin of replication); dsRNA (the genes EVO-SF-06 (SEQ ID 1), EVO-HA-09 (SEQ ID 2) and EVO-SF-10 (SEQ ID 3)); Ter (Terminator containing a stop codon to finalize the expression the dsRNA). In cosmid backbone: ColE1 ori (origin of replication in the phage); X^{r} (selection site for any type of selective marker); cos site (cosmid replication site).

FIG. 4—Stability, in days, of the molecule EVO-SF-06 (SEQ ID 1, dsRNA) submitted to ultraviolet (UV) light, maintained in 10 mM MOPS, pH 8.0. dsRNA molecule cloned into EVO-206910 and uncloned. The molecule integrity was measured in nanodrop.

FIG. 5—Stability, in days, of the molecule EVO-HA-09 (SEQ ID 2, dsRNA) submitted to ultraviolet (UV) light, maintained in 10 mM MOPS, pH 8.0. dsRNA molecule cloned into EVO-206910 and uncloned. The molecule integrity was measured in nanodrop.

FIG. 6—Stability, in days, of the molecule EVO-SF-10 (SEQ ID 3, dsRNA) submitted to ultraviolet (UV) light, maintained in 10 mM MOPS, pH 8.0. dsRNA molecule cloned into EVO-206910 and uncloned. The molecule integrity was measured in nanodrop.

FIG. 7—Stability, in days, of the molecule EVO-SF-06 (SEQ ID 1, dsRNA) submitted to ultraviolet (UV) light and mixed with surfactants for adherence on the leaf surface, maintained in 10 mM MOPS, pH 8.0. dsRNA molecule cloned into EVO-206910 and uncloned. Assays maintained at 35° C. in dry bath incubator. Molecule integrity measured in nanodrop. Clon_D1: cloned dsRNA+sodium lauryl ether sulphate (30%)+lecithin (10%), maintained under natural light and room temperature. Clon_D1_30_35: cloned dsRNA+lauryl sodium sulfate ether (30%)+lecithin (10%), UV for 30 days, maintained at 35° C. Clon_D1_35: cloned dsRNA+sodium lauryl ether sulphate (30%)+lecithin (10%), maintained under natural light and temperature of 35° C. Non-clon_D1_35_30: uncloned dsRNA+sodium lauryl ether sulphate (30%)+lecithin (10%), UV for 30 days, maintained at 35° C. Clon_D2: cloned dsRNA+sodium lauryl ether sulphate (20%)+lecithin (5%), maintained under natural light and room temperature. Clon_D2_30_35: cloned dsRNA+sodium lauryl ether sulphate (20%)+lecithin (5%), UV for 30 days, maintained at 35° C. Clon_D2_35: cloned dsRNA+sodium lauryl ether sulphate (20%)+lecithin (5%), maintained under natural light and temperature of 35° C. Non-clon_D2: uncloned dsRNA+sodium lauryl ether sulphate (20%)+lecithin (5%), maintained under natural light and room temperature. Non-clon_D2_35_30: uncloned dsRNA+sodium lauryl ether sulphate (20%)+lecithin (5%), UV for 30 days, maintained at 35° C.

FIG. 8—Stability, in days, of the molecule EVO-HA-09 (SEQ ID 2, dsRNA) submitted to ultraviolet (UV) light and mixed with surfactants for adherence on the leaf surface, maintained in 10 mM MOPS, pH 8.0. dsRNA molecule cloned into EVO-206910 and uncloned. Assays maintained at 35° C. in dry bath incubator. Molecule integrity measured in nanodrop. Clon_D1: cloned dsRNA+sodium lauryl ether sulphate (30%)+lecithin (10%), maintained under natural light and room temperature. Clon_D1_30_35: cloned dsRNA+lauryl sodium sulfate ether (30%)+lecithin (10%), UV for 30 days, maintained at 35° C. Clon_D1_35: cloned dsRNA+sodium lauryl ether sulphate (30%)+lecithin (10%), maintained under natural light and temperature of 35° C. Non-clon_D1_35_30: uncloned dsRNA+sodium lauryl ether sulphate (30%)+lecithin (10%), UV for 30 days, maintained at 35° C. Clon_D2: cloned dsRNA+sodium lauryl ether sulphate (20%)+lecithin (5%), maintained under natural light and room temperature. Clon_D2_30_35: cloned dsRNA+sodium lauryl ether sulphate (20%)+lecithin (5%), UV for 30 days, maintained at 35° C. Clon_D2_35: cloned dsRNA+sodium lauryl ether sulphate (20%)+lecithin (5%), maintained under natural light and temperature of 35° C. Non-clon_D2: uncloned dsRNA+sodium lauryl ether sulphate (20%)+lecithin (5%), maintained under natural light and room temperature. Non-clon_D2_35_30: uncloned dsRNA+sodium lauryl ether sulphate (20%)+lecithin (5%), UV for 30 days, maintained at 35° C.

FIG. 9—Stability, in days, of the molecule EVO-SF-10 (SEQ ID 3, dsRNA) submitted to ultraviolet (UV) light and mixed with surfactants for adherence on the leaf surface, maintained in 10 mM MOPS, pH 8.0. dsRNA molecule cloned into EVO-206910 and uncloned. Assays maintained at 35° C. in dry bath incubator. Molecule integrity measured in nanodrop. Clon_D1: cloned dsRNA+sodium lauryl ether sulphate (30%)+lecithin (10%), maintained under natural light and room temperature. Clon_D1_30_35: cloned dsRNA+lauryl sodium sulfate ether (30%)+lecithin (10%), UV for 30 days, maintained at 35° C. Clon_D1_35: cloned dsRNA+sodium lauryl ether sulphate (30%)+lecithin (10%), maintained under natural light and temperature of 35° C. Non-clon_D1_35_30: uncloned dsRNA+sodium lauryl ether sulphate (30%)+lecithin (10%), UV for 30 days, maintained at 35° C. Clon_D2: cloned dsRNA+sodium lauryl ether sulphate (20%)+lecithin (5%), maintained under natural light and room temperature. Clon_D2_30_35: cloned dsRNA+sodium lauryl ether sulphate (20%)+lecithin (5%), UV for 30 days, maintained at 35° C. Clon_D2_35: cloned dsRNA+sodium lauryl ether sulphate (20%)+lecithin (5%), maintained under natural light and temperature of 35° C. Non-clon_D2: uncloned dsRNA+sodium lauryl ether sulphate (20%)+lecithin (5%), maintained under natural light and room temperature. Non-clon_D2_35_30: uncloned dsRNA+sodium lauryl ether sulphate (20%)+lecithin (5%), UV for 30 days, maintained at 35° C.

### DETAILED DESCRIPTION OF THE INVENTION

The mechanism of the interference RNA (RNAi) is a highly conserved post-transcriptional regulation mechanism in eukaryotes, which controls the expression of a gene by the level of mRNA expression within the cells, being triggered by double-stranded RNA (dsRNA).

According to the researchers, the sensitivity and response to RNAi treatment is variable for each order and species of insect, due to physiological characteristics, life habitats, feeding mechanisms and digestion, metabolic pathways, requiring optimization of the technique for each species. Among the most important diseases of agriculture is the soybean rust caused by Phakopsora pachyrhizi whose selected targets genes express the proteins NADS, COX1, RPS3, membrane transporter and gene that expresses for the cytochrome c oxidase protein. For white mold disease in soybeans, the target genes express enolase-phosphatase E1, methylthioribulose-1-phosphate dehydratase, vacuolar ATase and vacuolar membrane protease. For Cescospora disease in maize, the most important are gamma actin, beta tubulin, gene that expresses the mating protein type 1:1 and the gene for thioredoxin (4A). The same can be done for weeds by inactivating genes related to energy transport, carboxylase/oxygenase (chloroplast), vacuolar H+ATPase, photosystem II, among others.

Nozawa SR found that this sensitivity is related to the level of mRNA expression for each expressed gene. That is, if a gene has a lower level of expression, the sensitivity to inactivate it in a metabolic pathway is much higher than when compared to a high mRNA expression of a gene.

Therefore, the fine tuning for this technology to work is directly related to the level of gene expression that one wants to control the expression.

In this sense, here is described a process of selection of a nucleotide sequence for use as a phytosanitary product that comprises the following steps:
a. analysis of the transcriptome of the target pest;
b. selection of sequences of a low-expression gene, which are more sensitive to silencing, but which have participation in essential metabolic pathways, physiological and/or reproductive processes;
c. determination of homology and similarity of the selected sequences (in step b) with non-target organisms, and
d. selection of the target nucleotide sequence through a Decision Tree algorithm to identify the sequence of high metabolic performance and low similarity or homology with non-target organisms.

For the purposes of definition, “transcriptome analysis”, as is known to a technician in the subject, is the analysis of the expression of messenger RNA (mRNA) or its complementary DNA (cDNA) is subject to the verification of their levels of expression and function, both at the cellular level and in the physiology/morphology of the target pest as a whole.

Low-expression genes are those that produce few copies of mRNA and that are lost or degraded throughout transcription and translation, reaching a limited number of copies of protein or even a single mRNA. The choice of low-expression target genes allows a small number of dsRNA molecules to be enough for the inactivation of these genes or their transcription.

Furthermore, “essential metabolic pathways” means any metabolic pathway that has a significant impact (in this case, negative or limiting) on the survival and/or reproduction of the target pest, either by physiological pathways or morphological changes in the target pest or its descendants.

As is already known by a technician in the subject, the term “homology” should be understood as the degree of similarity between, in this case, nucleotide sequences of different organisms. “Similarity” means the degree of similarity between two sequences, considering not only identical matches, but also those that result in substitutions considered as conservative.

Transcriptome analysis can be performed through known and widely disseminated databases in the case that the target is a pest already known and studied from a genetic point of view.

In the case of a pest whose data are not yet available, a preliminary analysis of its transcriptome is then necessary to verify the expression and function of the sequences present in it.

Once the transcriptome study is complete, low expression sequences are selected, which are more sensitive to silencing, but have participation in essential metabolic pathways, physiological and/or reproductive processes.

It is essential that the silencing of the target sequence results in the death/debilitation of the target pest or impediment/decrease of its reproductive capacity in order to have effective pest control.

Once the candidate sequences are selected, their homology or similarity with non-target organisms is verified through traditional bioinformatics tools, which are available to a technician in the subject. This stage aims to avoid cross-silencing of genes in non-target organisms, for example, in the target insect to be controlled and in a beneficial insect of the fauna present in the environment of the treated crop or neighborhoods.

Therefore, in a preferred embodiment, sequences resulting in a degree of homology less than 15% related to non-target species are selected.

In an even more preferred embodiment, sequences resulting in a degree of homology less than 1% related to other non-target species are selected.

In a preferred embodiment, the degrees of homology are at least in relation to the taxonomic gender of the target to be controlled.

In an even more preferred embodiment, this degree of homology is at least in relation only to the species to be controlled.

It was also found that the size of the selected sequence is an important factor for the effective silencing of the gene of interest.

The size of the sequences to be used will depend on the variability of the target genes and on the results of bioinformatics analysis. The smaller the fragment size, the more difficult the inactivation through dsRNA, however, the inactivation becomes more gene specific. The larger the dsRNA fragment, the greater the chance of inactivation of homologous genes in other organisms (off-target effect). In this case, the fragment size will be determined by bioinformatics analysis that will determine the amplitude of the dsRNA size, varying from wider ranges to narrower ranges.

Therefore, the size of the dsRNA sequence may vary depending on the analysis and complexity of the genome of the organism in question. Unlike the one usually applied for RNAi, which consists of short RNA fragments, on average 15 to 25 nucleotides, in this invention the RNA to be produced by the expression of the target nucleotide sequence has a length between 20 and 500 nucleotides, preferably between 100 and 500 nucleotides, even more preferably between 200 and 300 nucleotides.

The RNA molecule, preferably a double-stranded RNA (dsRNA), activates the cutting mechanism of this RNA in small double-stranded RNAs (siRNA) by the action of the Dicer enzyme (ribonuclease III). Then, one of the siRNA strands is removed and the remaining strand is incorporated into a protein complex called RNA Induced Silencing Complex (RISC). Finally, the RISC complex identifies and degrades messenger RNA (mRNA) that have the same sequence as siRNA, preventing protein synthesis.

Therefore, the use of long dsRNA sequences increases the chances of silencing the target gene. However, in parallel, the chances of similarity with gene sequences that are not intended to be silenced, including non-target organisms, also increase.

Thus, in order to avoid this problem, bioinformatics algorithms are used to determine the percentages of homology or identity, parallel to each inclusion/removal of nucleotides from the candidate sequence, in order to ensure that the degrees of homology or identity remain within the acceptable range.

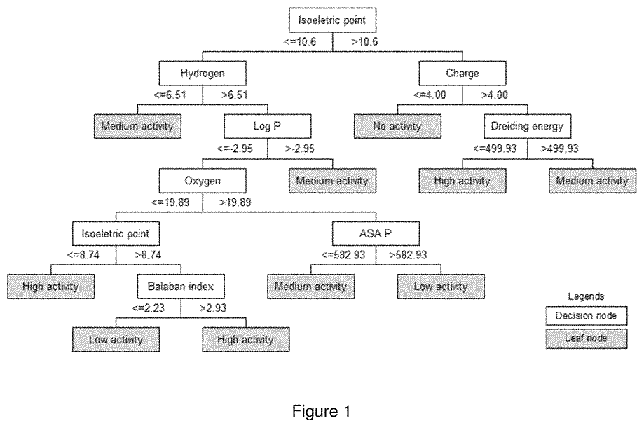

Once the candidate sequences are identified, the selection of the best sequence to be used as a phytosanitary product is made through a Decision Tree algorithm that comprises a machine learning algorithm, as shown in FIG. 1.

Studies by Lira, Baranauskas and Nozawa (2013) have shown, for antimicrobial peptides, that induction of Decision Trees is a machine learning approach that has been applied to various tasks. Decision Trees (DT) are suitable for large real-world tasks because they adapt well and can represent complex concepts, building simple but robust logic-based classifiers that can be directly interpreted by experts.

Top-down inductions of Decision Tree algorithms often choose a feature that partitions training data according to some evaluation functions. Partitions are recursively partitioned until some stop criterion is met. After that, the Decision Tree is removed to avoid overfitting.

In this case, Weka's J48 algorithm was used, a library of several machine learning algorithms. J48 is an open source Java implementation of the C4.5 algorithm available in the Weka package. However, as it is promptly conceived by a technician in the subject, any alternative algorithm can be used, provided that the criteria adopted lead to the detection of a sequence that meets the parameters described here.

In this case, the training data were composed of 60 target DNA sequences with low expression in the genome that express functional proteins, each described by 53 molecular descriptors. These descriptors include structure, liquid load, hydrophobic residues, and Bowman index, among others, and were obtained using the Marvin Beans (www.chemaxon.com/download/marvin) program package.

Single-stranded RNA (ssRNA) polynucleotides were divided into four classes according to their microbial activity (none, low, medium and high), as follows. A specific dsRNA was classified as “none” if no activity was found in any of the cell types, “low” if the activity occurred in only one organism, “average” if the activity occurred in exactly two organisms and “high” if it occurred in three or more organisms.

According to this procedure, the distribution of dsRNA in classes none, low, medium and high in the training data was 3 (1%), 13 (9%), 19 (15%), respectively. To select the most predictive attributes and find the best parameter setting for J48, we used the technique called “windowing”, in which the Decision Tree model begins to learn with only a fraction (window) of the examples in the dataset.

A classifier is induced using the initial window and is tested using examples that are not present in the window. A fraction of the examples outside the window, which were incorrectly sorted, are added to the window.

A new classifier is induced and tested, and the process is repeated until there are no classification errors. The window can be repeated several times (attempts), starting with a different start window each time. The window provided by C4.5 was used.

After applying the technique with different C4.5 and window settings, the tree was chosen with the best test error.

Then, a new data set was constructed, composed only of the attributes found in the unpruned version of the best tree: liquid load, hydrogen, oxygen, isoelectric point, accessible surface area to peptides (ASA_P), Balaban index, reduction energy, minimum projection radius and the logarithm ratio of the partition coefficient [log (P)].

Using Weka, J48 was executed on the dataset with the filtered attributes. The standard parameters for the inductor were used, except for the M parameter, which determines the minimum number of examples that a sheet must contain. A value for Mof 3 was used, with which the best previously found tree was defined.

This same methodology was used for the selection of amino acid sequences with high metabolic activity performance. The result is a highly selective, exclusive peptide that can be synthesized in the laboratory for inhibition or deletion of expression via RNA, preferably dsRNA.

Once the nucleotide sequence is selected, it is inserted into a plasmid or cosmid, by conventional techniques widely known by a technician in the subject.

To modulate their correct expression, in a preferred embodiment, the selected sequences are assembled with at least promoter and terminator regions, which are compatible with the expression promoters of the cell in which the plasmid or cosmid will be inserted.

In a preferred embodiment, this plasmid or cosmid additionally comprises genes or regions responsible for its replication, genes or regions responsible for its replication in the phage and/or genes or selection regions.

In a preferred embodiment, this gene or region of selection comprises one or more genes or regions capable of selecting transfected cells with plasmid or cosmid described here.

In an even more preferred embodiment, this gene or region of selection comprises one or more genes or sites of antibiotic or antifungal resistance, such as the ampicillin resistance gene (Amp^{r}).

Several selection genes can be placed in the plasmid, such as genes for resistance to tetracycline, kanamycin, quinolones, heavy metals, lactose operon, hygromycin, phosphinothricin, glyphosate, neomycin phosphotransferase, hygromycin phosphotransferase, phosphonothricin acetyltransferase, glyphosate oxy-reductase, Isopentenyl transferase, benzyladenine N-3-glucuronide, phosphomannose-isomerase, D-serine dehydratase, D-amino acid oxidase, among others.

Therefore, a host cell comprising at least one copy of this plasmid or cosmid is also described.

In a preferred embodiment, this host cell is selected from a bacterium or yeast cell.

In an even more preferred embodiment, this host cell is an Escherichia coli cell.

Other types of host cells can be used, such as, but not limited to, the selected group of Picchia pastoris, Neurospora crassa, Saccharomices cerevisae, Aspergillus nidulans, Saccharomyces ssp, Bacillus ssp, among others.

In an even more preferred embodiment, the genetically modified L4440 cosmid is used.

Another embodiment describes a process for producing an RNA comprising the following steps:
a. selection of a nucleotide sequence, according to the method described above;
b. preparation of a plasmid or cosmid, as defined above;
c. transfection of a host cell with this plasmid;
d. multiplication of host cells in a bioreactor, and
e. expression of the selected nucleotide sequence.

In a preferred embodiment, the RNA expressed in the step (e) of the above process is an interference RNA (RNAi).

In an even more preferred embodiment, the RNA expressed in the step (e) of the above process is a double-stranded RNA (dsRNA).

Thus, it is also described an RNA molecule that is obtained by the above process, preferably an RNAi, more preferably a dsRNA.

In a preferred embodiment, this RNA molecule is for use as a phytosanitary product.

Therefore, it is also described the use of this RNA molecule as a phytosanitary product to eliminate or reduce the infestation of an insect, disease or weed.

Using the example of corn, the second crop (winter corn) has increased significant in the western region of Parana, replacing other crops, because it becomes a crop of greater economic viability. It is a wide-ranging crop that can be grown on small, medium and large farms, where the level of investment can vary according to the way the farmer will manage his crop (Richetti & Ceccon, 2011). However, the succession of same crops favors the occurrence of the main diseases (Juliatti et al., 2009).

In the past, the control of diseases in corn was traditionally done using varieties with greater resistance, together with farming measures. However, in recent years, pesticide use has become mandatory to protect crop yield, so great emphasis has been given to the use of fungicides. The use of agrochemicals to control diseases in corn is very recent, and there are doubts on the part of technicians and growers in various aspects of their use, which often results in the inappropriate use of the products, without observing the necessary technical aspects.

Runoff is one of the most important risk factors of agrochemical applications. It is a complex process, influenced by several factors, such as the type of spraying equipment used and the physicochemical properties of the spray solution (Broniarz-Press, 2009). By runoff, the sprayed product can reach unwanted sites, causing environmental and socioeconomic impacts, reaching the neighborhood and plantations near the sprayed crops (Chechetto, 2011).

The effectiveness of a phytosanitary treatment, especially for contact-action products, requires perfect coverage of the biological target, usually leaves, stems, flowers and fruits, so that this coverage is highly influenced by climatic factors (temperature, air humidity, dew, wind, luminosity) and by characteristics of the formulated product (solubility, stability, density, viscosity, volatilization, foam, surface tension, among others).

In this context, the use of adjuvants in the spray solution is mandatory, providing better spreading of the product on the target surface (lost, 2010).

The excipients (adjuvants) added to fungicides provide greater efficiency to the protection of leaves against pathogens, due to the increased spread of the sprayed drop on the leaf surface, even under unfavorable environmental conditions, reducing the volume of the application, reducing waste and preserving the environment.

According to Mendonça, (2007), excipients (adjuvants) can improve the efficiency of applications, but the interaction between adjuvant and agrochemical is a complex process that involves many physical, chemical and physiological aspects, and can vary for each condition tested.

The dosage and formulation of adjuvants interfere in the physicochemical properties of the spray solutions to be used, including the spectrum of droplet sizes. The surface tension, viscosity and pH are the most sensitive properties to the addition of adjuvants.

The pH defines the degree of alkalinity or acidity of a solution, on a scale from 0 to 14, where 7 means neutrality. Pure water has pH 7, but pH is frequently altered by various dissolutions. Many chemicals, when prepared with water, suffer degradation, so the pH can influence the stability of the active ingredient.

The products are formulated to tolerate some variability in the pH of the spray solution. However, extreme values can affect physical stability. According to Kissmann (1997), pH can influence the outcome of the application. When the pH of the water is high, it can accelerate the degradation of the phytosanitary products by alkaline hydrolysis and its absorption by plant tissues can vary, depending on the molecule being whole or dissociated into cations and anions.

According to Wanamarta & Penner (1989), the leaf surface has neutral pH, but interaction with the pH of the spray solution can occur.

Moreover, it is known that genetic material (DNA or RNA) is sensitive to environmental exposures, being subject to degradation by several factors such as pH, ultraviolet (UV) light, temperature, among others.

As seen above, it is essential that a stabilized compound be provided in order to ensure that the RNA molecule described here is preserved on the leaf surface until its ingestion or absorption by the target organism (insect, disease or weed), so that the phytosanitary effect is achieved.

This stabilized compound is achieved by combining the RNA molecule with one or more stabilizing agents.

The patent documents PI0509743-6, PI0706266-4, PI0613235-9 and BR112013009803-1 report the potential of RNAi for insect or arachnid control, which is widely known and published in scientific papers around the world. However, these documents do not mention any method of stabilization and manufacture of RNAi molecules, which is critical for the action of these molecules in crop conditions for pest control and, therefore, for the viability of their commercial use by farmers as a phytosanitary product.

This invention covers all the characteristics necessary for the use of RNAi as a phytosanitary product, that is, efficacy of control of the target species, molecule stability for more than 40 days on the leaf surface and large-scale production of this active ingredient.

Keeping dsRNA molecules adhered to leaf surfaces is a challenge for the scientific community that manipulates and/or develops dsRNA. The dsRNA formulations prepared for laboratory testing purposes are completely different to the formulation to be used under crop conditions, in terms of resistance to high temperatures, rates and types of solvents and other components.

The current document demonstrates the action of dsRNA in the open field environment with high incidences of UV radiation and high temperatures in relation to different types of protective and spread-adhesive solutions. The solutions presented in the patent documents PI0509743-6, PI0706266-4, PI0613235-9 and BR112013009803-1 do not simulate the reality of corn, soybean or cotton planting fields and may also cause breakage, rupture and/or denaturation of the double-strand of ribonucleic acid (dsRNA) destabilizing the molecule and, therefore, drastically affecting the efficacy to control the target pest.

In a preferred embodiment, stabilizing agents are selected from the group comprising sodium lauryl ether sulphate and lecithin.

Other classes of stabilizing agents can be used in this process provided that ideal concentrations are calibrated, such as anionic (alkylbenzene sulfonate, sodium dodecyl sulfate, sodium n-lauroyl sarcosinate), non-ionic (polyethylene glycols, alcohols, alkylphenols and ethoxylated amines), cationic (quaternary ammonium salts such as cetylpyridinium chloride, dodecyltrimethylammonium chloride, hex dodecyl benzyl methyl ammonium chloride), amphoteric (betaines), among others.

Single and combined excipients (adjuvants), available in the market, can be used in the RNA spray solution to efficiently spread on the leaf surface, such as nonylphenoxypoly (ethyleneoxy) ethanol, soybean oil methyl ester, vegetable oil, polyether copolymer, polyether-silicone copolymer, mineral oil, among others.

Lecithin was used for emulsification, but other classes of products could be used following the rules of surface tension.

The static surface tension of aqueous solutions with mineral oils and vegetable oils was evaluated according to the methodology described by Mendonça et al. (1999). This property was evaluated in each solution containing registered products for farming use, like mineral oils and vegetable oils.

The concentrations of oils used to determine the surface tension of aqueous solutions were: 0.025; 0.05; 0.1; 0.25; 0.5; 0.75; 1.0; 1.5; 2.0; 2.5 and 3.0% v/v.

The static surface tension of the solutions was estimated by measuring the mass of 15 drops, formed at the tip of a burette placed in an analytical scale, with an accuracy of 0.01 mg. Each measured drop corresponds to a replicate. The environmental temperature was 25° C.±2° C.

The analysis of variance and regression were determined to the set of data obtained for each product, adjusting these data to the Mitscherlich Model. The analysis of variance and regression was performed by the SAS program. For the Mitscherlich Model to fit the data, there was a need to modify it. The original and simplified templates are presented below.

Original Model: Y=A(1−10^{-C(X+B)}).

Simplified Used Model: Y=T_{water}−A(1−10^{-CX}),
where:
Y—surface tension, mN·m⁻¹;
A—maximum horizontal asymptote in the original model;
C—concavity of the curve;
B—point of interception of the abscissa axis;
T_{water} —72.6 mN·m⁻¹;
X—oil % concentration;
(T_{water}−A)—minimum horizontal asymptote in simplified model.

All elements of the equation have practical meaning: the expression “T_{water}−A” corresponds to the minimum surface tension that an oil can reach from the concentration at which it reaches this surface tension value and even if increasing the concentration of the oil in question, we do not obtain decreases in surface tension values. Parameter “C” represents the efficiency of the product (emulsifiable oil). The higher the value of this parameter, the more efficient the oil to reach the minimum surface tension at a lower concentration. Parameter “B” were not used in this model, since no product mixtures were used. This parameter represents how much surfactant (emulsifiable oil) must be added in order to obtain the same tension reduction conditioned by the addition of the other product, such as an herbicide, at a certain concentration.

The pH ranges depend directly on the type of biological buffer used in the system and should be kept within an ideal range to maintain the molecule stability with high performance.

In a preferred embodiment, the stabilized compound additionally comprises a pH above 6.8 which is maintained using a buffer.

In an even more preferred embodiment, the pH is kept in the range between 6.8 and 8.7.

In a preferred embodiment, the buffer used in the stabilized compound was selected from the group of 3-Morpholino-2-hydroxypropanesulfonic acid (MOPS).

Nozawa SR conducted a test with the dsRNA molecules EVO-SF-06 (SEQ ID 1), EVO-HA-09 (SEQ ID 2) and EVO-SF-10 (SEQ ID 3) to measure its stability under ultraviolet (UV) light with the use of 10 mM MOPS, pH of 7.5 to 8.5. The stabilized and non-stabilized molecules were submitted to UV light for up to 45 days and the readings were made every 5 days in nanodrop.

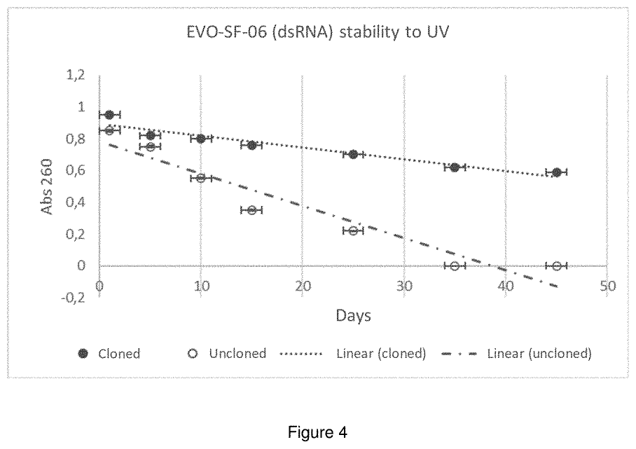

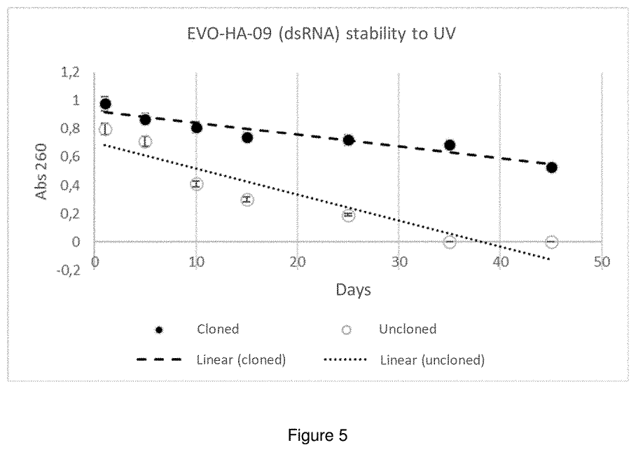

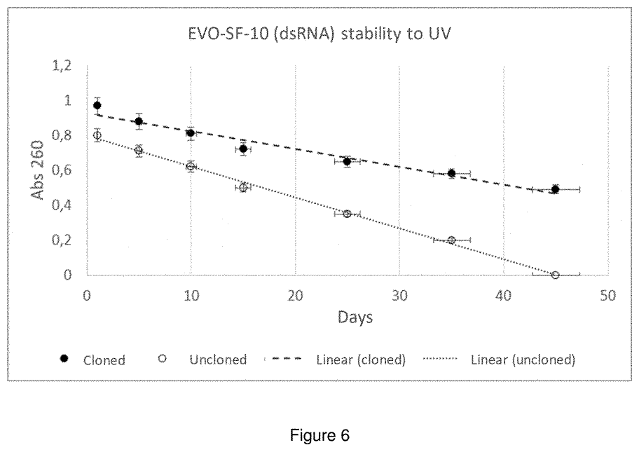

The evaluated results, presented in the FIGS. 4 to 6, demonstrate the buffering efficiency provided by MOPS, since all stabilized molecules (cloned in the cosmid EVO-206910) remained stable (intact) under ultraviolet radiation types A, B and C, as a function of a given exposure period. All graphs show that UV radiation affects the double-strand of ribonucleotides and destabilizes uncloned molecules, making them degradable, whereas when they are cloned in the cosmid EVO-206910, the circular molecules are stable when exposed to UV light (at any distance and dosage/day). The small observed loss of activity of dsRNA molecules in cosmid is due to the tendency of the cosmid to form circular/spiral structures that make it difficult to read in nanodrop, although they are in fact stabilized.

Other buffers can be used if tested for buffering efficiency, molecule activity and the resulting pH.

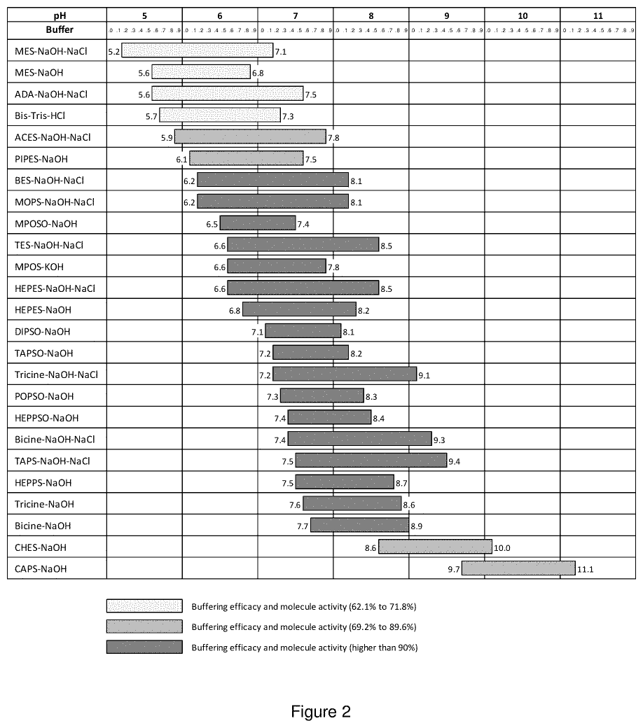

As an example, Nozawa SR conducted a test to measure buffering efficiency, molecule activity, and resulting pH. Twenty-five different buffers were tested, 17 of which provided buffering efficiency and molecule activity above 90% (FIG. 2) among them:
BES (N,N-bis(2-hydroxyethyl)-2-aminoethanesulfonic acid)-NaOH—NaCl,
MOPS (3-(N-morpholino)propanesulfonic acid)-NaOH—NaCl,
MOPSO (3-Morpholino-2-hydroxypropanesulfonic acid)-NaOH,
TES (2-[[1,3-dihydroxy-2-(hydroxymethyl)propan-2-yl]amino]ethanesulfonic acid)-NaOH—NaCl,
MOPS (3-(N-morpholino)propanesulfonic acid)-KOH,
HEPES (4-(2-hydroxyethyl)-1-piperazineethanesulfonic acid)-NaOH—NaCl,
HEPES (4-(2-hydroxyethyl)-1-piperazineethanesulfonic acid)-NaOH,
DIPSO (N,N-Bis(2-hydroxyethyl)-3-amino-2-hydroxypropanesulfonic acid)-NaOH,
TAPSO (2-Hydroxy-3-[tris(hydroxymethyl)methylamino]-1-propanesulfonic acid)-NaOH,
Tricine (N-[Tris(hydroxymethyl)methyl]glycine)-NaOH—NaCl,
POPSO (Piperazine-1,4-bis(2-hydroxypropanesulfonic acid) dihydrate)-NaOH,
HEPPSO (4-(2-Hydroxyethyl)piperazine-1-(2-hydroxypropanesulfonic acid)-NaOH,
Bicine (N,N-Bis(2-hydroxyethyl)glycine)-NaOH—NaCl,
TAPS (N-[(1S,2S,3R)-2,3-bis(acetyloxy)-1-[(acetyloxy)methyl]heptadecyl]-acetamide)-NaOH—NaCl,
HEPPS (4-(2-Hydroxyethyl)-1-piperazinepropanesulfonic acid)-NaOH,
Tricine (N-[Tris(hydroxymethyl)methyl]glycine)-NaOH,
Bicine (N,N-Bis(2-hydroxyethyl)glycine)-NaOH.

Additionally, a phytosanitary product is described here, which comprises an RNA molecule or a stabilized compound.

In a preferred embodiment, the phytosanitary product also comprises one or more agronomically acceptable excipients (adjuvants) for better spread on the leaf surface, which are selected, but not restricted, to the group comprising vehicles, surfactants, stabilizers and emulsifiers. All vehicles, surfactants, stabilizers and emulsifiers can be used in the formulation, such as nonylphenoxypoly (ethyleneoxy) ethanol, soybean oil methyl ester, vegetable oil, polyether copolymer, polyether-silicone copolymer, mineral oil, among others.

Finally, a method is described to eliminate or reduce the infestation of an insect, disease or weed in the crop, which includes spraying this phytosanitary product over the infested area or the leaf surface.

### Examples

Synthesis of dsRNA

As an example, the synthesis of dsRNA was performed according to the method described by Tenllado et al. (2003). Initially, recombinant cosmids were extracted from E. coli XL1-Blue and used for transformation into electrocompetent cells of E. coli HT115 (DE3).

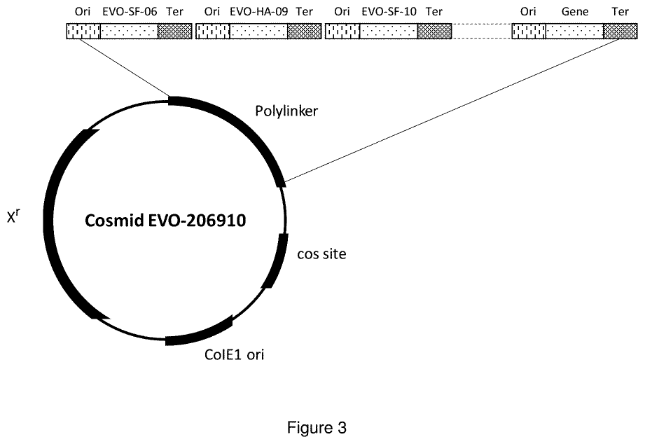

For this test, the cosmid EVO-206910 was used, as shown in FIG. 3.

One colony of E. coli HT115 (DE3) containing recombinant cosmid was added to 2 mL of LB liquid medium plus ampicillin (50 μg·mL⁻¹) and tetracycline (12.5 μg·mL⁻¹) under stirring (250 rpm) at 37° C. After approximately 16 h, 500 μL of the LB medium containing bacterial cells were added to 25 mL of LB liquid medium, with the antibiotics mentioned above, under stirring (250 rpm) at 37° C., until reaching a concentration of 7×10⁻⁸ cells/mL estimated by the turbidity index by spectrophotometry. Then, the bacteria were induced to produce dsRNA by adding 0.4 mM of IPTG and incubated at 37° C. under stirring of 250 rpm for 3 h. After inducing the production of dsRNA, total nucleic acids extraction of bacteria was performed, as described by Timmons et al. (2001).

The cells were collected by centrifugation at 1,500×g for 10 min (4° C.) and resuspended in 800 μL of extraction solution (1 M ammonium acetate, 10 mM EDTA) and equal phenol:chloroform volume (1:1; v:v) at 65° C., followed by vigorous stirring for 1 min.

The material was transferred to 1.5 mL microcentrifuge tubes and centrifuged at 18,000×g for 10 min (4° C.).

The upper phase was transferred to a new tube, adding 2.5×the volume of absolute ethanol to precipitate the material for 1 hour at −70° C., followed by centrifugation at 16,000×g for 20 min (4° C.).

The upper phase was discarded, and the precipitate was washed with 1 mL of 70% ethanol. A new centrifugation was performed at 16,000×g for 5 min (4° C.), and the material was resuspended in dilution solution (10 mM Tris-HCl, pH 7.5, 1 mM EDTA).

DsRNA was visualized by electrophoresis in 1% agarose gel, followed by staining in ethidium bromide (0.5 μg·mL⁻¹).

One DNA standard (1 kb DNA Ladder Invitrogen) was included in each gel, which were photographed as previously described.

The presence of double-stranded of the material was confirmed by incubation with RNase A, which degrades single-stranded RNA (ssRNA). The reaction (10 μL) was composed of 36 ng of RNA, 0.1 μg of RNase A enzyme and 1 μL of 7.5 M ammonium acetate, with incubation at 37° C. for 1 hour.

The reaction products were visualized by electrophoresis in 2% agarose gel, followed by ethidium bromide staining (0.5 μg·mL⁻¹). One DNA standard (1 kb DNA Ladder Invitrogen) was included in each gel.

For the production of dsRNA in a 2 L STR type bioreactor (Setric Genie Industriel), the cells were cultured in modified mineral salts, using the best condition provided in factorial design (2.5% (w/v) wheat bran and 1.2% (w/v) corn starch).

The resulting strain was cultured in 1000 mL of culture medium (pH 6.8), inoculated with/mL of standardized spore suspension (4×10⁹ CFU/mL).

The effect of the variables stirring speed and aeration rate variables on dsRNA production was studied using 2×2 factorial planning.

Fermentation was conducted for 144 hours (6 days), and every 24 hours, a 15 mL aliquot was sampled and the total volume was centrifuged (4,000 rpm/4° C.) and filtered, and the raw supernatants were frozen (−20° C.) for further analysis.

The pH and dissolved oxygen parameters were monitored throughout the fermentation process and the experiments were conducted in duplicate.

dsDNA was formulated in a compound comprising sodium lauryl ether sulphate and lecithin at different concentrations, maintaining the pH above 7.5 with the use of MOPS buffer.

Stability of dsRNA Molecules with the Use of MOPS

Nozawa SR conducted a test with the dsRNA molecules EVO-SF-06 (SEQ ID 1), EVO-HA-09 (SEQ ID 2) and EVO-SF-10 (SEQ ID 3) to measure its stability under ultraviolet (UV) light with the use of 10 mM MOPS, pH of 7.5 to 8.5. The stabilized and non-stabilized molecules were submitted to UV light for up to 45 days and the readings were made every 5 days in nanodrop.

The evaluated results, presented in FIGS. 4 to 6, demonstrate the buffering efficiency provided by MOPS, since all stabilized molecules (cloned in cosmid EVO-206910) remained stable (intact) under ultraviolet radiation types A, B and C, as a function of a given exposure period. All graphs show that UV radiation affects the double-strand of ribonucleotides and destabilizes uncloned molecules, making them degradable, whereas when they are cloned in the cosmid EVO-206910, circular molecules are stable when exposed to UV rays (at any distance and dosage/day). The small observed loss of activity of dsRNA molecules in cosmid is due to the tendency of the cosmid to form circular/spiral structures that make it difficult to read in nanodrop, although they are in fact stabilized.

Stability of dsRNA Molecules with the Use of MOPS, Lauryl Sodium Sulfate and Lecithin

Nozawa SR conducted a test with the dsRNA molecules EVO-SF-06 (SEQ ID 1), EVO-HA-09 (SEQ ID 2) and EVO-SF-10 (SEQ ID 3) to measure its stability after different periods of exposure to ultraviolet (UV) light. The dsRNA molecules were cloned in the cosmid EVO-206910, maintained in 10 mM MOPS (pH 8.0), at a temperature of 35° C. (in dry bath) and added sodium lauryl ether sulphate and lecithin, at concentrations of 30%+10% and 20%+5%. The integrity (stability) readings were made in nanodrop, every 10 days of exposure to UV, up to 40 days.

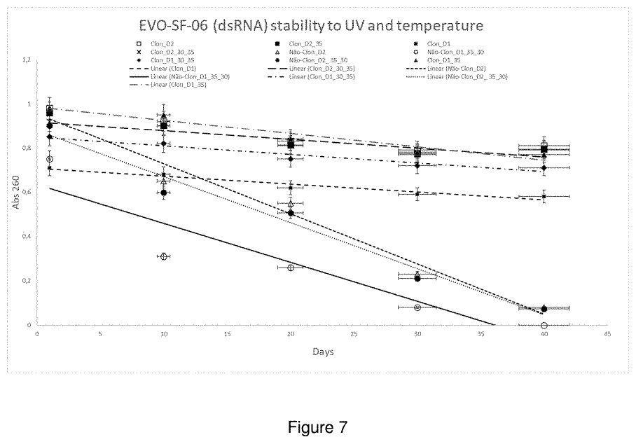

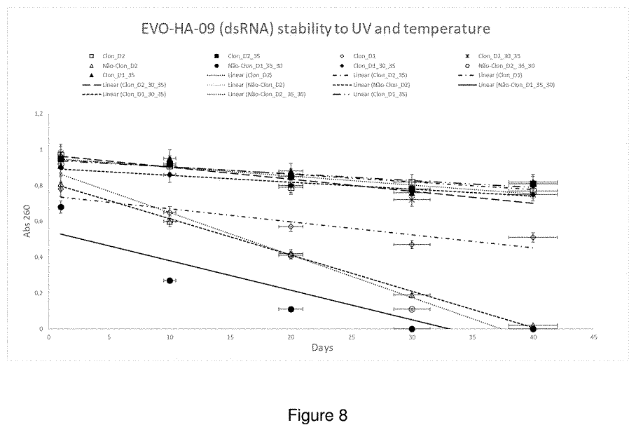

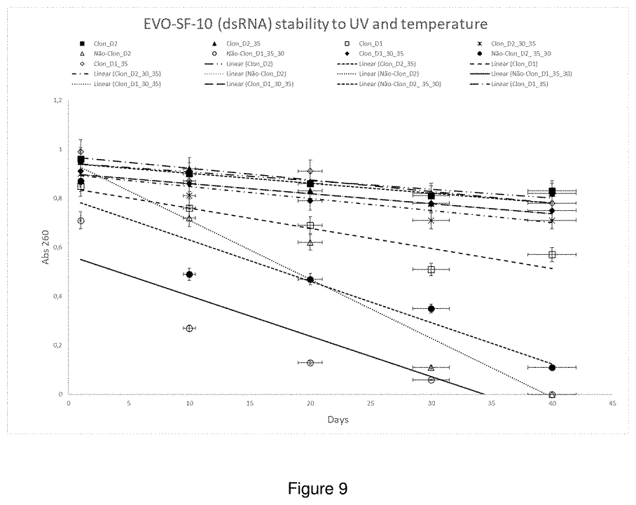

The evaluated results, presented in the FIGS. 7 to 9, demonstrate that the addition of sodium lauryl ether sulphate and lecithin, in the tested concentrations, did not affect the stability of cloned dsRNA molecules, even under UV light and high temperatures, conditions expected in farming areas. As shown in FIGS. 4 to 6, there is a protection and stabilization of dsRNA molecules when cloned in the cosmid EVO-206910, so that the addition of lauryl sulfate sodic and lecithin surfactants did not affect both stability and activity, not even with the increase in room temperature (25° C. in CNTP) up to 35° C. in thermostat incubator. The uncloned dsRNA molecules were affected by high temperatures and UV radiation in all evaluated periods. High temperatures and incidence of UV rays destabilize the double-strand of the uncloned dsRNA, making it unsuitable for use under the typical farming environmental conditions.

As shown in FIGS. 4 to 9, the RNA molecules in the stabilized compounds showed significantly improved stability when compared to non-stabilized molecules.

Efficacy of Stabilized dsRNA Molecules for the Control of Spodoptera frugiperda and Helicoverpa armigera

Studies were conducted by an Entomology Company to evaluate the efficacy of stabilized dsRNA molecules EVO-SF-06 (SEQ ID 1), EVO-HA-09 (SEQ ID 2) and EVO-SF-10 (SEQ ID 3) to control of Spodoptera frugiperda and Helicoverpa armigera, served by ingestion (in the diet) and spraying (topical application). The results evaluated in 4 bioassays showed that, after 10 days, the tested products in all methods of administration (ingestion, spraying, spraying+ingestion) caused effects on larval development and protein synthesis, demonstrated by instar changing, growth inhibition, color change, behavior alteration, among others, leading to a disruption of target metabolic pathways and leading to mortality of Spodoptera frugiperda and Helicoverpa armigera, at levels technically acceptable (above 60%), regardless of the tested dsRNA doses, which ranged from 0.12 μg up to 1.2 μg. It was also observed that the time for mortality of the tested species did not depend on the dose applied. The molecules, therefore, were site-specific and species-specific.

After describing the examples of preferred achievements, the scope of this invention covers other possible variations, being limited only by the content of the claims, including the possible equivalents.

### REFERENCES

Broniarz-Press L.; Ochowiak, M.; Rozanski, J.; S. Woziwodzki, S. (2009). The atomization of water-oil emulsions, Experimental Thermal and Fluid Science, Volume 33, Issue 6, 2009. Pages 955-962.
Burtet, L. M.; Bernardi, O.; Melo, A. A.; Pes, M. P.; Strahl, T. T., Guedes, J. V. (2017). Managing fall armyworm, Spodoptera frugiperda (Lepidoptera: Noctuidae), with Bt maize and insecticides in southern Brazil. Pest Manag. Sci. 73(12): 2569-2577. http://dx.doi.org/10.1002/ps.4660.
Chechetto, R. G (2011). Potencial de redução da deriva em função de adjuvantes e pontas de pulverização. 2011. viii, 70 f. Dissertação (mestrado)—Universidade Estadual Paulista, Faculdade de Ciências Agronômicas de Botucatu, 2011.
EPPO (2018). Data sheets on Quarantine Pests Spodoptera frugiperda. Data Sheets on Quarantine Pests. E. P. P. organization, EPPO.
Farias, R.; Horikoshi, R. J.; Santos, A. C.; Omoto, C. (2014). Geographical and Temporal Variability in Susceptibility to Cry1F Toxin from Bacillus thuringiensis in Spodoptera frugiperda (Lepidoptera: Noctuidae) Populations in Brazil. J. Econ. Entomol. 107(6): 2182-2189. http://dx.doi.org/10.1603/EC14190.
Goergen, G.; Kumar, P. L.; Sankung, S. B.; Togola, A.; Tamò, M. (2016). First Report of Outbreaks of the Fall Armyworm Spodoptera frugiperda (J E Smith) (Lepidoptera, Noctuidae), a New Alien Invasive Pest in West and Central Africa. PLoS ONE 11(10): e0165632. https://doi.org/10.1371/journal.pone.0165632.
Iost, C. A. R; Raetano, C. G. (2010). Tensão superficial dinâmica e ângulo de contato de soluções aquosas com surfactantes em superficies artificiais e naturais. Engenharia Agrícola, 30(4), 670-680. https://dx.doi.org/10.1590/S0100-69162010000400011.
IRAC. (2019). Comitê de Ação à Resistência a Inseticidas. Projetos. Spodoptera frugiperda. Disponível em https://www.irac-br.org/Spodoptera-frugiperda. Acessado em 30 de agosto de 2019.
IRAC-BR. (2013). Comitê Brasileiro de Ação a Resistência a Inseticidas. Manejo da Resistência de Spodoptera frugiperda a Inseticidas e Plantas Bt. Autores: Celso Omoto, Odeleri Bernardi, Eloisa Salmeron, Juliano Ricardo Freitas. ESALQ/USP, Piracicaba, SP.
Juliatti, F. C., Pedrosa, M. G., Silva, H. D. (2009). Genetic mapping for resistance to gray leaf spot in maize. Euphytica 169, 227-238 (2009). https://doi.org/10.1007/s10681-009-9943-2
Kissmann K. G.; Groth, D. (1997). D. Plantas infestantes e nocivas. São Paulo: BASF Brasileira, 1997. p. 675-678. Tomo I.
Lira, F.; Perez, P. S.; Baranauskas, J. A.; Nozawa, S. R. Nozawa. (2013). Prediction of Antimicrobial Activity of Synthetic Peptides by a Decision Tree Model. Applied and Environmental Microbiology. April 2013, 79 (10) 3156-3159; DOI: 10.1128/AEM.02804-12.
Mendonça, F.; Danni-Oliveira, I. M. (2007). Climatologia: noções básicas e climas do Brasil. São Paulo: Oficina de Texto, 2007. 206 p.
Omoto, C.; Bernardi, O.; Salmeron, E.; Sorgatto, R. J.; Dourado, P. M.; Crivellari, A.; Carvalho, R. A.; Willse, A.; Martinelli, S.; Head, G. P. (2016). Field-evolved resistance to Cry1Ab maize by Spodoptera frugiperda in Brazil. Pest Manag Sci. 72(9):1727-36.
Richetti, A. (2011). Viabilidade econômica do sistema de produção soja milho safrinha. In: Seminário Nacional de Milho Safrinha, 11, 2011, Lucas do Rio Verde. De safrinha a grande safra: anais. Lucas do Rio Verde: Fundação Rio Verde: ABMS, 2011. 524-530.
Tenllado, F., Martínez-García, B., Vargas, M. et al. (2003). Crude extracts of bacterially expressed dsRNA can be used to protect plants against virus infections. BMC Biotechnol 3, 3 (2003). https://doi.org/10.1186/1472-6750-3-3.
Timmons L.; Court, D. L; Fire, A. (2001). Ingestion of bacterially expressed dsRNAs can produce specific and potent genetic interference in Caenorhabditis elegans, Gene, Volume 263, Issues 1-2, 2001, Pages 103-112.
Wanamarta, G.; Penner, D. Foliar absorption of herbicides (1989). Weed Sci., v. 4, n. 1, p. 215-232, 1989.

1. Process of selection of a nucleotide sequence for use as a phytosanitary product, characterized by the fact that it comprises the following stages:
a. analysis of the transcriptome of the target pest;
b. selection of sequences of a low-expression gene, which are more sensitive to silencing, but which have participation in essential metabolic pathways, physiological and/or reproductive processes;
c. determination of homology and similarity of sequences selected in step (b) with other non-target organisms,
d. selection of the target nucleotide sequence through a Decision Tree algorithm to identify the sequence of high metabolic performance and low similarity or homology with non-target organisms.

2. Process of selection of a nucleotide sequence, according to claim 1, characterized by the fact that homology or similarity with non-target organisms is less than 30%, preferably less than 5%.

3. Process of selection of a nucleotide sequence, according to claim 1, characterized by the fact that the selected nucleotide sequence has a length between 20 and 500 nucleotides, preferably between 100 and 500 nucleotides, even more preferably between 200 and 300 nucleotides.

4. Process of selection of a nucleotide sequence, according to claim 1, characterized by the fact that the Decision Tree algorithm consists of a machine learning algorithm.

5. Plasmid or cosmid, characterized by the fact that it comprises at least one copy of the nucleotide sequence selected by the process as defined in claim 1.

6. Plasmid or cosmid, according to claim 5, characterized by the fact that it additionally comprises promoter and terminator regions for expression of the selected nucleotide sequence.

7. Plasmid or cosmid, according to claim 5, characterized by the fact that it additionally comprises genes for plasmid or cosmid replication, genes for replication in the phage and/or selection genes.

8. Plasmid or cosmid, according to claim 7, characterized by the fact that the selection gene comprises one or more antibiotic or antifungal resistance genes, such as genes for resistance to ampicillin, kanamycin, hygromycin, phosphinothricin, neomycin, quinolone, among others.

9. Host cell characterized by the fact that it comprises at least one plasmid or cosmid as defined in claim 6.

10. Host cell, according to claim 9, characterized by the fact that it is a cell of bacteria or yeast.

11. Host cell, according to claim 9, characterized by the fact that it is a selected cell of the group of Escherichia coli, Saccharomyces ssp, Bacillus ssp, Picchia pastoris, Neurospora crassa, among others.

12. Process to produce an RNA, characterized by the fact that it comprises the following steps:
a. selection of a nucleotide sequence, as defined in claim 1;
b. preparation of a plasmid or cosmid, characterized by the fact that it comprises at least one copy of the nucleotide sequence selected by the process as defined in claim 1;
c. transfection of a host cell, with this plasmid or cosmid;
d. multiplication of host cells in a bioreactor; and
e. expression of the selected nucleotide sequence.

13. Process to produce an RNA, according to claim 12, characterized by the fact that RNA is an interference RNA (RNAi).

14. Process for producing an RNA, according to claim 12, characterized by the fact that RNA is a double-stranded RNA (dsRNA).

15. RNA molecule characterized by the fact that it is obtained by the process as defined in claim 12.

16. RNA molecule, according to claim 15, characterized by the fact that it is an RNAi.

17. RNA molecule, according to claim 15, characterized by the fact that it is a dsRNA.

18. RNA molecule, according to claim 15, characterized by the fact that for use as phytosanitary product.

19. Use of an RNA molecule, as defined in claim 15, characterized by the fact that it is as a phytosanitary product to eliminate or reduce the infestation of an insect, disease or weed in crops.

20. Stabilized compound, characterized by the fact that it comprises an RNA molecule, as defined in claim 15, in combination with one or more stabilizing agents.

21. Stabilized compound, according to claim 20, characterized by the fact that stabilizing agents are selected from the group comprising anionic stabilizing agents (alkylbenzene sulfonate, sodium dodecyl sulfate, sodium n-lauroyl sarcosinate), non-ionic (polyethylene glycols, alcohols, alkylphenols and ethoxylated amines), cationic (quaternary ammonium salts such as cetylpyridinium chloride, dodecyltrimethylammonium chloride, hex dodecyl benzyl methyl ammonium chloride), amphoteric (betaines), sodium lauryl ether sulphate and lecithin, among others.

22. Stabilized compound, according to claim 20, characterized by the fact that it has a pH above 6.8, preferably a pH between 6.8 to 8.7 and a buffer.

23. Stabilized compound, according to claim 22, characterized by the fact that the buffer is any buffer that maintains the buffering efficiency and the activity of the RNA molecule, in which the buffer is preferably selected from, but not limited to, BES (N,N-bis(2-hydroxyethyl)-2-aminoethanesulfonic acid)-NaOH—NaCl, MOPS (3-(N-morpholino)propanesulfonic acid)-NaOH—NaCl, MOPSO (3-Morpholino-2-hydroxypropanesulfonic acid)-NaOH, TES (2-[[1,3-dihydroxy-2-(hydroxymethyl)propan-2-yl]amino]ethanesulfonic acid)-NaOH—NaCl, MOPS (3-(N-morpholino)propanesulfonic acid)-KOH, HEPES (4-(2-hydroxyethyl)-1-piperazineethanesulfonic acid)-NaOH—NaCl, HEPES (4-(2-hydroxyethyl)-1-piperazineethanesulfonic acid)-NaOH, DIPSO (N,N-Bis(2-hydroxyethyl)-3-amino-2-hydroxypropanesulfonic acid)-NaOH, TAPSO (2-Hydroxy-3-[tris(hydroxymethyl)methylamino]-1-propanesulfonic acid)-NaOH, Tricine (N-[Tris(hydroxymethyl)methyl]glycine)-NaOH—NaCl, POPSO (Piperazine-1,4-bis(2-hydroxypropanesulfonic acid) dihydrate)-NaOH, HEPPSO (4-(2-Hydroxyethyl)piperazine-1-(2-hydroxypropanesulfonic acid)-NaOH, Bicine (N,N-Bis(2-hydroxyethyl)glycine)-NaOH—NaCl, TAPS (N-[(1S,2S,3R)-2,3-bis(acetyloxy)-1-[(acetyloxy)methyl]heptadecyl]-acetamide)-NaOH—NaCl, HEPPS (4-(2-Hydroxyethyl)-1-piperazinepropanesulfonic acid)-NaOH, Tricine (N-[Tris(hydroxymethyl)methyl]glycine)-NaOH, Bicine (N,N-Bis(2-hydroxyethyl)glycine)-NaOH.

24. Phytosanitary product, characterized by the fact that it comprises an RNA molecule, as defined in claim 15, or a stabilized compound characterized by the fact that it comprises an RNA molecule, as defined in claim 15, in combination with one or more stabilizing agents.

25. Phytosanitary product, according to claim 24, characterized by the fact that it comprises one or more selected excipients, but not restricted to the group comprising vehicles, surfactants, stabilizers and emulsifiers, in which excipients are preferably selected from nonylphenoxypoly (ethyleneoxy) ethanol, methyl ester of soybean oil, vegetable oil, polyether copolymer, polyether-silicone copolymer, mineral oil, among others.

26. Method to eliminate or reduce the infestation of an insect, disease or weed in crop, characterized by the fact that it consists of spraying, on the infested area or the leaf surface, a phytosanitary product as defined in claim 24.
